(19) United States
(12) Patent Application Publication
(10) Pub. No.: US 2009/0211573 A1
(43) Pub. Date: Aug. 27, 2009

(54) ENDOTRACHEAL TUBE HOLDER WITH IMPROVED LOCKING DEVICE

(76) Inventor: Ronald D. Russo, Barrington, RI (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/035,847

(22) Filed: Feb. 22, 2008

(51) Int. Cl. A61M 16/00

(52) U.S. Cl. 128/200.26; 128/207.14; 128/207.17

(57) ABSTRACT

An endotracheal tube holder assembly is provided. The holder assembly comprises a support strap band for placement around a patient's head or neck region and a flexible linking band for attachment to the support strap band. The flexible linking band further comprises an elongate, central bridge portion, an upper bar portion extending from the central bridge portion, and at least one tab extending laterally from the central bridge portion.

### TECHNICAL FIELD

The present disclosure relates to the field of medical device holders, and more specifically, to methods and systems for securing endotracheal tubes to a patient.

### BACKGROUND

Endotracheal tubes are commonly used to ventilate patients for resuscitation, anesthesia, and other critical care procedures. Endotracheal tubes help deliver air to patients who cannot breathe on their own due, for example, to injury, trauma, stroke, or illness. Once inserted, it is desirable to secure the endotracheal tube in a fixed position to prevent movement or extubation of the tube from the patient's airway.

A number of methods have been used to secure endotracheal tubes. For example, an endotracheal tube can be secured to a patient by affixing the tube to the patient's cheek or face using adhesive tape. Further, in some cases twill ties can be used, either alone or in combination with adhesive tape.

Typically, endotracheal tube holders fall into three main categories: tape type products, mechanical screw or compression strap type holders, and bite block type holders. Examples of these are tape devices as described, for example in U.S. Pat. No. 5,638,814 to Byrd and U.S. Pat. No. 5,797,394 to Boyd; screw or compression type devices as described in U.S. Pat. No. 5,490,504 to Vrona et al., U.S. Pat. No. 4,548,200 to Wapner, and U.S. Pat. No. 5,513,633 to Islave; and bite-block type devices as described in U.S. Pat. No. 3,946,742 to Eross.

Although these methods of securing endotracheal tubes can be effective, these methods may have some shortcomings. For example, commercially-available adhesive tape-type products for use with endotracheal tube holders tend to sag and lift off the patient's face after limited use. Further, because tape must be removed frequently for suctioning and repositioning, considerable skin irritation and possible infection can occur. In addition, endotracheal tube securing devices that incorporate a bite-block are also available, but such devices have not received widespread acceptance since bite-blocks tend to irritate the patient's mouth and tongue after a short period of use.

In addition, endotracheal tubes are often connected to heavy, bulky, breathing circuitry along with a closed tracheal suction catheter, which tends to place significant tension on associated endotracheal tube securing devices. Also, humidifiers, water condensate traps, and oxygen lines, which may be part of the breathing circuit, add weight and tension exerted on the endotracheal tube, and can further cause such tubes to loosen or move. Purely adhesive tape-type products cannot withstand the constant load strain produced by such devices, thereby leading to tube movement, sagging, or kinking. These types of misplacement or deformation of the tube can cause the airway to become partially shut off and can result in the loss or reduction of administered ventilation to the patient.

### SUMMARY

In one exemplary embodiment consistent with principles of the present disclosure, the holder assembly comprises a support strap band for placement around a patient's head or neck region and a flexible linking band for attachment to the support strap band. The flexible linking band further comprises an elongate, central bridge portion, an upper bar portion extending from the central bridge portion, and at least one tab extending laterally from the central bridge portion.

Another illustrative embodiment includes a method for securing an endotracheal tube in a patient's mouth. This can include placing a support strap band around the patient's head or neck region and securing the support strap band to an endotracheal tube. The step of securing the support strap band to an endotracheal tube can include providing a flexible linking band including an elongate, central bridge portion attached to the support strap band, an upper bar portion extending from the central bridge portion, and at least one tab extending laterally from the central bridge portion. Further, the method includes the step of wrapping the at least one tab around the endotracheal tube.

### BRIEF DESCRIPTION OF THE DRAWINGS

The accompanying drawings, which are incorporated in and constitute a part of this specification, provide exemplary embodiments of the disclosure and, together with the description, serve to explain the principles of the disclosure.

FIG. 1 illustrates an unassembled perspective view of a tube holder assembly, according to one exemplary embodiment.

FIG. 2 illustrates an unassembled perspective view of a tube holder assembly, according to another exemplary embodiment.

FIG. 3 illustrates an assembled perspective view of the tube holder assembly of FIG. 1, according to one exemplary embodiment.

FIG. 4 illustrates a frontal view of the tube holder assembly of FIG. 1 attached to the endotracheal tube, according to one exemplary embodiment.

FIG. 5 illustrates a perspective view of the tube holder assembly of FIG. 1 depicting diametrically opposing tabs securing an endotracheal tube on a patient, according to one exemplary embodiment.

FIG. 6 illustrates a partial perspective view of diametrically opposing tabs on the holder assembly of FIG. 1 depicting the application of inward compressive force of the tabs on an endotracheal tube, according to one exemplary embodiment.

FIG. 7 illustrates an assembled perspective view of a tube holder assembly, according to another exemplary embodiment.

FIG. 8 illustrates a cross-sectional view of tube holder assembly material, according to one exemplary embodiment.

FIG. 9 illustrates a rolled perspective view of linking band material, as may be used to produce the tube holder assemblies of the present disclosure.

### DETAILED DESCRIPTION OF THE DRAWINGS

An improved holder assembly is disclosed herein for securing a medical tube, such as for example, an endotracheal tube, to a patient. The assembly includes a self gripping holder for securing an endotracheal tube. The holder assembly allows positioning of an endotracheal tube without the need for tape, screw compression means, or bite blocks.

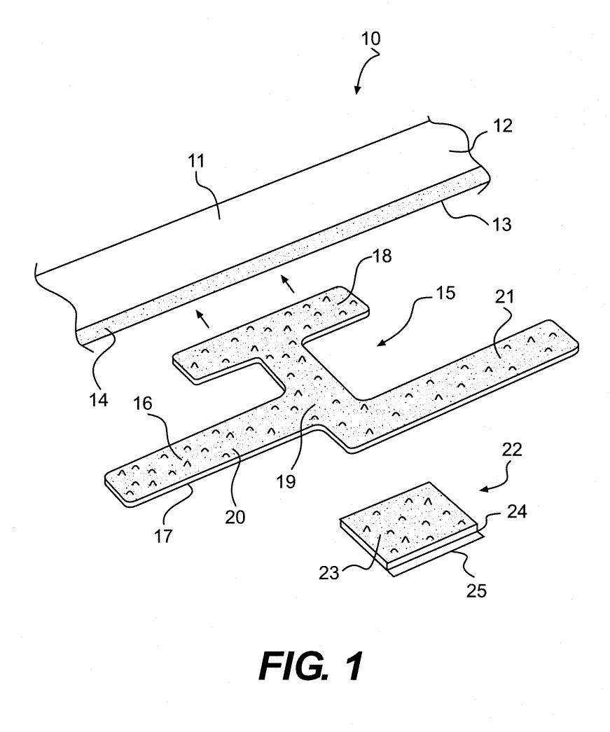

FIG. 1 illustrates an unassembled perspective view of a tube holder assembly 10, according to one exemplary embodiment. As shown, tube holder 10 can include a support strap band 11 configured to encircle a head or neck region of a patient to secure the assembly 10 to the patient. The assembly 10 can further include a flexible linking band 15 for securing the support strap band 11 to an endotracheal tube, as described in detail below. In some embodiments, the linking band 15 can include an upper bar portion 18 having a central bridge 19 from which two opposing tabs 20, 21 extend. Further, in some embodiments, the holder assembly 10 can further include a strip portion 22, configured to facilitate reversible attachment of the linking band 15 to an endotracheal tube, as described further below.

The support strap band 11 can be made from a soft, comfortable yet strong material such as Velcro™ loop pile fabric, which is flexible and comfortable. The support strap band 11 can have a top surface 12 and an opposing bottom surface 13 both made from Velcro™ loop pile fabric with a soft breathable polyurethane foam core 14 in between. The support strap band 11 is typically one inch wide and about thirty inches long, and can terminate in Velcro™. However, the support strap band 11 can be a range of sizes as the dimensions of the support strap band 11 can be adjusted according to the size of the patient. For example, for smaller children, the support strap band 11 can be less than one inch in width and less than thirty inches in length. For larger patients, the support strap band 11 can be greater than one inch in width and greater than thirty inches in length. The support strap band 11 can also terminate in adhesive material (not shown) for securement behind the patient's head or neck.

The ends of the support strap band 11 can include complementary mating elements that can interlock to hold the ends together. In one embodiment, the support strap band 11 can terminate in Velcro™ hook type closures for securement behind the patient's head or neck. In another embodiment, the support strap band 11 can terminate in adhesives for securement behind the patient's head or neck.

As noted, the holder assembly 10 can include a flexible linking band 15 for securing the support strap band 11 to an endotracheal tube. As shown, in one embodiment, the flexible linking band 15 can include a top Velcro™ hook surface 16 and a bottom Velcro™ loop surface 17. Further, the linking band 15 can include an upper bar portion 18 configured to be attached to the support strap band 11. In some embodiments, the upper bar portion 18 can be attached to the support strap band 11 by stitches, Velcro™, adhesives, or with combinations of such attachment systems.

In some embodiments, the linking band 15 will further include a central bridge 19 extending from the bar portion 18. The central bridge 19 can include at least one tab that extends laterally from the central bridge 19 and can be wrapped around an endotracheal tube to secure the tube in place, as described below. In one embodiment, as shown in FIG. 1, the at least one tab can include two opposing tabs 20, 21, and the two opposing tabs 20, 21 can be positioned at varying distances from the upper bar portion 18 and extend laterally from the central bridge 19 so that when they are wrapped around the endotracheal tube, they do not overlap one another. Further, as shown, in one embodiment, one tab 20 of the at least two laterally extending tabs extends in a first direction relative to the central bridge 19, and another tab 21 of the at least two laterally extending tabs extends in a direction substantially opposite the first direction relative to the central bridge 19. In one embodiment, the tabs 20, 21 are longer than the circumference of an endotracheal tube to be secured by the holder assembly 15 so that when they are wrapped around the endotracheal tube, they can mate onto themselves. For example, if the tabs are configured so that the Velcro™ hook surface is on top of the tab and the loop surface is on the bottom of the tab, when the tab is wrapped around the endotracheal tube, the bottom loop surface 17 can mate with the top hook surface 16.

As noted, the holder assembly 10 can further include a small Velcro™ adhesive-backed strip 22 having a hook top surface 23, a lower adhesive surface 24, and an extended release liner 25. The strip 22 is typically made from Velcro™ molded plastic hook material, although other materials can be used. For example, this material may be Velcro™ molded plastic hook material HTH833. The extended release liner 25 can cover the lower adhesive surface 24 and can be removed to expose the lower adhesive surface 24 of the strip 22. In one embodiment, the hook top surface 23 of the strip 22 can be removably attached to the bottom Velcro™ loop surface 17 of the flexible linking band 15, and the lower adhesive surface 24 can be attached to the endotracheal tube.

The removable attachment between the adhesive strip 22 and linking band 15 allows removable but secure attachment between the holder assembly 10 and an endotracheal tube. For example, the adhesive strip 22 can provide a fixed attachment that is unlikely to slide or otherwise allow movement of an endotracheal tube. Further, the removable connection to the linking band 15 allows the tube holder assembly 10 to be temporarily disengaged when needed.

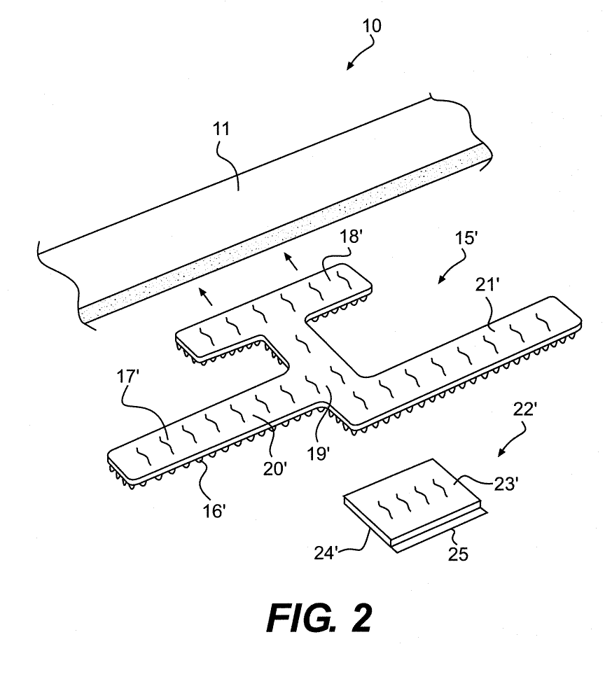

FIG. 2 depicts an alternative embodiment of an unassembled endotracheal tube holder 10. As shown, the tube holder 10 has the same support strap band 11, but the linking band 15′ has a top loop surface 17′ and a bottom hook surface 16′. The linking band 15′ comprises an upper bar portion 18′ having a central bridge 19′ from which two opposing tabs 20′, 21′ extend. In one embodiment, the two opposing tabs 20′, 21′ can be at varying distances from the upper bar portion 18′ and extend laterally from the central bridge 19′ so that when they are wrapped around the endotracheal tube, they do not overlap each other. Further, the adhesive loop strip 22′ has a lower adhesive surface 24′ with an extended release liner 25 for attachment to the endotracheal tube and an upper loop pile material surface 23′ for attachment to the hook surface 16′ of the linking band 15′. The extended release liner 25 can cover the lower adhesive surface 24′ and can be removed to expose the lower adhesive surface 24′ of the strip 22′.

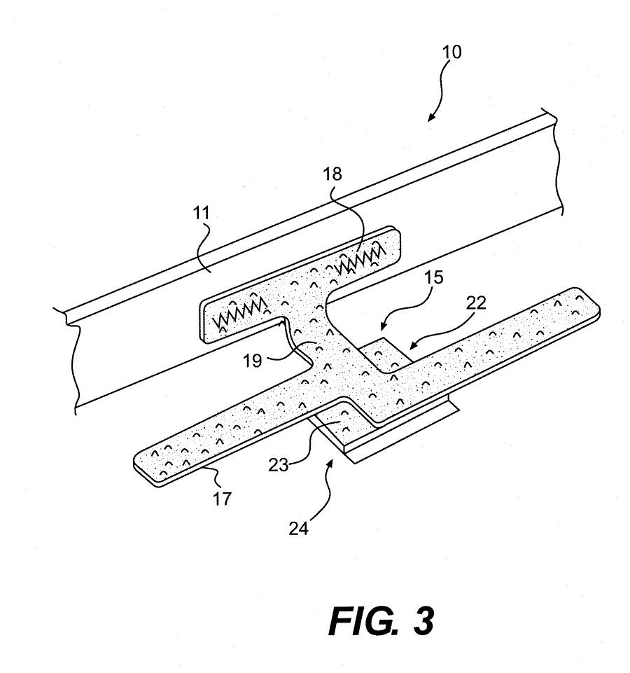

FIG. 3 illustrates the endotracheal tube holder 10 of FIG. 1 in its assembled form. As shown, the linking band 15 can be sewn onto the support strap band 11 at the upper bar portion 18, and the bridge portion 19 can bend or flex with respect to the upper bar portion 18. The adhesive strip 22, having hook top surface 23 can be pre-assembled and attached to the bottom Velcro™ loop surface 17 on the linking band 15. The lower adhesive surface 24 of the strip 22 can be attached to the endotracheal tube, as described in further detail below.

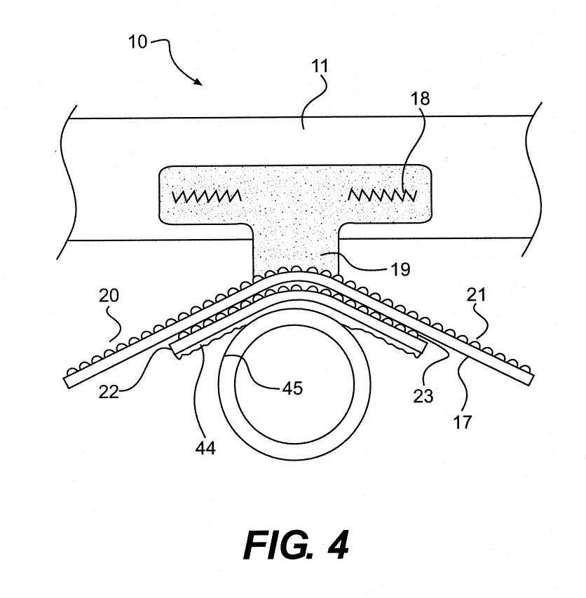

FIG. 4 illustrates a frontal view of the tube holder assembly of FIG. 1 attached to an endotracheal tube, according to one exemplary embodiment. As shown, the upper bar portion 18 is attached to the support strap band 11 and can flex upwards or downwards with respect to the support strap band 11. Further, the adhesive strip 22 is attached to the bottom Velcro™ loop surface 17 via the hook top surface 23.

As can be seen in FIG. 4, once the extended release liner 25 (shown in FIG. 1) is removed from the strip 22, it exposes a solvent-based adhesive 44 that can permanently or semi-permanently adhere to an endotracheal tube 45. The strip 22 can be wrapped around the endotracheal tube 45 so the solvent based adhesive 44 is no longer exposed, and the strip 22 is attached to the tube 45. The two opposing tabs 20, 21 can be wrapped around the endotracheal tube 45, thereby securing the tube holder 10 to the tube 45.

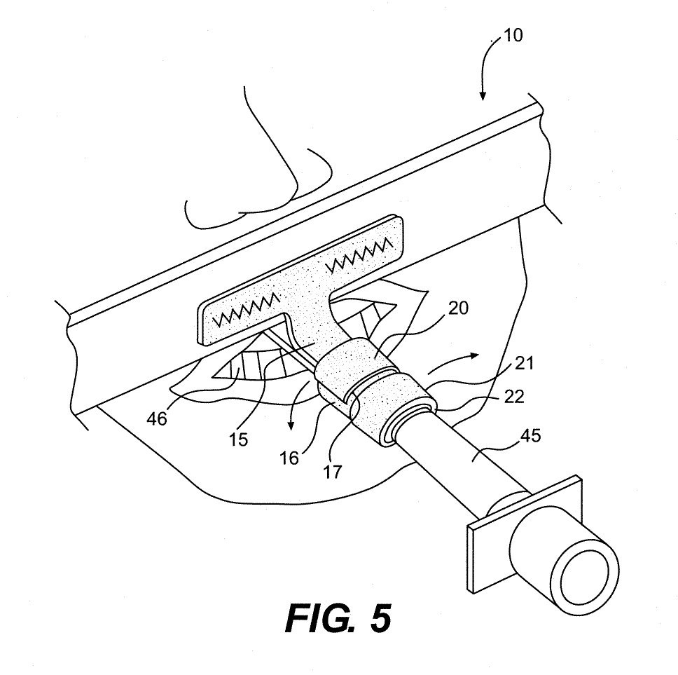

FIG. 5 illustrates a perspective view of the tube holder assembly 10 of FIG. 1 depicting diametrically opposing tabs securing an endotracheal tube 45 on a patient, according to one exemplary embodiment. Again, the linking band 15 is attached to the adhesive strip 22, which is attached to the endotracheal tube 45. The tabs 20, 21 of the linking band 15 can be wrapped around the endotracheal tube 45 and affixed via the Velcro™ loop surface 17 mating with the Velcro™ hook surface 16.

The stabilization of the endotracheal tube 45 by the tabs 20, 21 of the linking band 15 and the adhesive strip 22 can allow the endotracheal tube 45 to fit inside the patient's oral cavity 46. Further, with the tube holder assemblies of the present disclosure, a caregiver can place a suction tube in the patient's mouth to remove oral secretions. With the present disclosure, the suction tube can be easily moved in and out of the patient's mouth without disturbing the endotracheal tube 45.

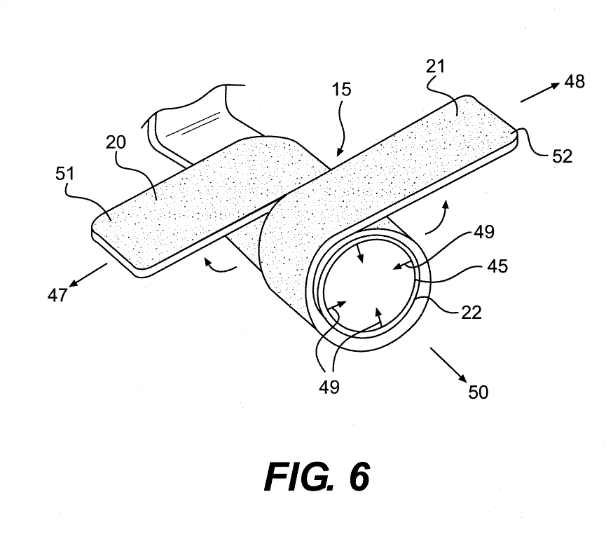

FIG. 6 further illustrates how the tabs 20, 21 of the linking band 15 can be pulled in tension 47, 48 by the user at end tab portions 51, 52 to produce a dual inward compressive force 49 on the endotracheal tube 45. The dual inward force 49 applied to the endotracheal tube 45 resists dislodgement or movement of the tube 45 when an outward pulling force 50 is applied to the endotracheal tube 45 from either the weight of attached ventilator circuitry or movement by disoriented or combative patients.

In addition, the self gripping tube holders of the present disclosure can be easily disengaged by lifting up on tab ends 51, 52 so that the entire linking band 15 and support strap band 11 can be replaced with a new holder. It should be noted that adhesive strip 22 can remain on the tube 45 for easy replacement of the same or another pre-assembled linking band 15 and support strap band 11.

As can be seen, the endotracheal tube holder 10 is easy to set up and use and allows stable positioning of the endotracheal tube 45, while providing complete access to the oral cavity for good oral care. The endotracheal tube holder 10 provides a soft touch engagement with the endotracheal tube 45, yet the engagement is extremely strong to resist tube pull out.

The endotracheal tube flow path is not crushed by the endotracheal tube holder 10, as is the case with screw type or compression band type holders, and there is no need for separate tape to secure the linking band 15. Tape is often difficult to apply to the linking band and the tape is difficult to remove from the linking band when the holder needs replacement.

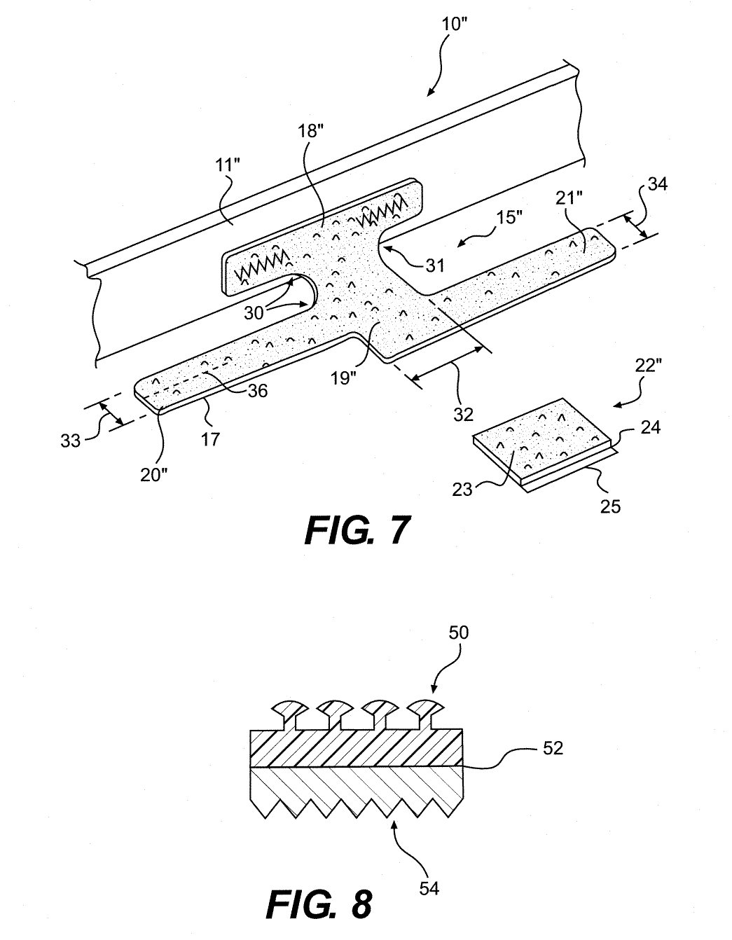

FIG. 7 illustrates an assembled perspective view of a tube holder assembly 10″, according to another exemplary embodiment. As shown, the tube holder 10″ again includes a support strap band 11″ configured to encircle a head or neck region of a patient to secure the assembly 10″ to the patient. The assembly 10″ can further include a flexible linking band 15″ for securing the support strap band 11″ to an endotracheal tube. In some embodiments, the linking band 15″ can include an upper bar portion 18″ having a central bridge 19″ from which two opposing tabs 20″, 21″ extend. Further, in some embodiments, holder assembly 10″ can further include a strip portion 22″, configured to facilitate reversible attachment of linking band 15″ to an endotracheal tube, as described further below.

As shown, the tube holder assembly 10″ can be configured to provide a bridge 19″ that is stiffer and resists lateral movement or flexure, thereby further stabilizing an endotracheal tube held by the assembly 10″. In one embodiment, the bar portion 18″ and bridge 19″ are connected at rounded sections 30, 31. For example, on one side, the bar portion 18″ and bridge 19″ are connected at a full radius 30 or semi-circular connection, and on the opposite side, are connected with a half radius 31 or quarter circle connection. These rounded connections help reduce lateral bending and flexure.

In addition, the bridge 19″ and tabs 20″, 21″ may be sized to further increase resistance to bending and flexure. For example, in one embodiment, the bridge 19″ may have a width 32 that is greater than the widths 33, 34 of either of tabs 20″, 21″. It will be understood that the widths of these components may vary based on the specific endotracheal tubes used, but typical widths can include about 0.625 inches for width 32 of bridge 19″, and about 0.425 inches for either or both of widths 33, 34 of tabs 20″, 21″.

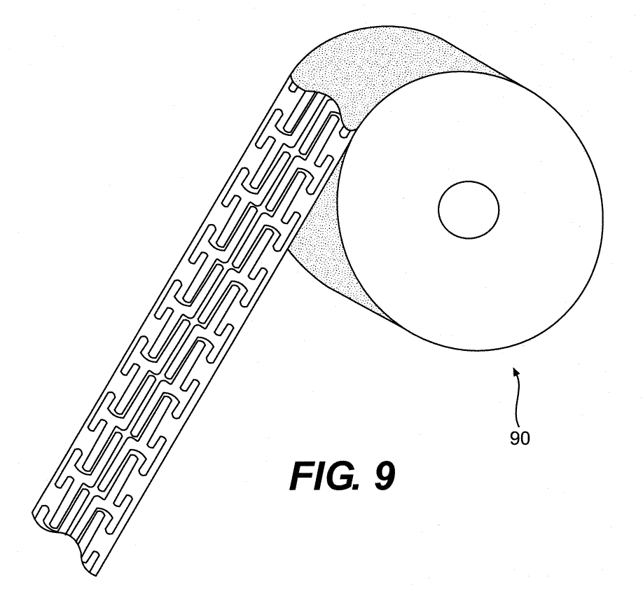

In addition, the material used for the disclosed tube assemblies may be selected to increase resistance to movement while securing the tube. As noted, in some embodiments, the tube assemblies of the present disclosure will include a laminated hook and loop material. FIG. 8 illustrates a cross-sectional view of the tube holder assembly of FIG. 7, according to one exemplary embodiment, illustrating the laminated hook and loop material through section 36. As shown, the material includes a semi-rigid laminate 52 with opposing hook 50 and loop material 54 layers.

The tube holder assemblies of the present disclosure may be produced using a variety of manufacturing techniques. For example, FIG. 9 illustrates a rolled perspective view of linking band material 90, as may be used to produce the tube holder assemblies of the present disclosure. As shown, the rolled linking band material 90 may be die cut to produce the desired assembly structure in a fast and efficient manufacturing process.

It will be apparent to those skilled in the art that additional various modifications and variations can be made consistent with the present disclosure. For example, various features within the several embodiments disclosed herein can be combined with features from other tube holder assembly embodiments. Other embodiments consistent with the tube holder will be apparent to those skilled in the art from consideration of the specification and practice of the disclosure. It is intended that the specification and examples be considered as exemplary only, with a true scope and spirit of the disclosure being indicated by the following claims.

1. A holder assembly for securing an endotracheal tube to a patient, the holder assembly comprising:
a support strap band for placement around a patient's head or neck region; and
a flexible linking band for attachment to the support strap band, the flexible link band comprising:
an elongate, central bridge portion;
an upper bar portion extending from the central bridge portion; and
at least one tab extending laterally from the central bridge portion.

2. The holder assembly of claim 1, wherein the central bridge portion comprises at least two laterally extending tabs.

3. The holder assembly of claim 2, wherein one of the at least two laterally extending tabs extends in a first direction relative to the central bridge, and another of the at least two laterally extending tabs extends in a direction substantially opposite the first direction relative to the central bridge.

4. The holder assembly of claim 3, wherein the two laterally extending tabs are located at different distances from the upper bar portion.

5. The holder assembly of claim 2, wherein the two laterally extending tabs are each longer than the circumference of an endotracheal tube to be secured by the holder assembly.

6. The holder assembly of claim 5, wherein the flexible linking band is affixed to the support strap band using at least one of stitches, sutures, and an adhesive.

7. The holder assembly of claim 1, wherein one end of the support strap band includes complementary mating elements to secure the support strap band to a patient.

8. The holder assembly of claim 7, wherein the complementary mating elements comprise hook and loop material.

9. The holder assembly of claim 7, wherein the complementary mating elements comprise an adhesive.

10. The holder assembly of claim 1, wherein the bridge portion has a width that is greater than a width of the at least one laterally extending tabs.

11. The holder assembly of claim 1, wherein the bridge portion is connected to the upper bar portion at substantially rounded connections on opposing sides of the bridge portion.

12. A holder assembly of claim 1, further comprising:
a strip having a first side configured to engage the flexible linking band and a second side including an adhesive configured to engage an endotracheal tube.

13. The holder assembly of claim 12, wherein the central bridge portion comprises at least two laterally extending tabs.

14. The holder assembly of claim 13, wherein one of the at least two laterally extending tabs extends in a first direction relative to the central bridge, and another of the at least two laterally extending tabs extends in a direction substantially opposite the first direction relative to the central bridge.

15. The holder assembly of claim 14, wherein the two laterally extending tabs are located at different distances from the upper bar portion.

16. The holder assembly of claim 13, wherein the two laterally extending tabs are each longer than the circumference of an endotracheal tube to be secured by the holder assembly.

17. The holder assembly of claim 12, wherein the flexible linking band is affixed to the support strap band.

18. The holder assembly of claim 12, wherein the bridge portion has a width that is greater than a width of the at least one laterally extending tabs.

19. The holder assembly of claim 12, wherein the bridge portion is connected to the upper bar portion at substantially rounded connections on opposing sides of the bridge portion.

20. The holder assembly of claim 10, wherein the strip first side includes at least one of a hook material and a loop material configured to engage a hook or loop material on a surface of the flexible linking band.

21. A method for securing an endotracheal tube in a patient's mouth, comprising:
placing a support strap band around the patient's head or neck region; and
securing the support strap band to an endotracheal tube, the method of securing comprising:
providing a flexible linking band including an elongate, central bridge portion attached to the support strap band, an upper bar portion extending from the central bridge portion, and at least one tab extending laterally from the central bridge portion; and
wrapping the at least one tab around the endotracheal tube.

22. The method of claim 21, further including providing a securing strip including a first side including at least one of a hook material and a loop material and a second side including an adhesive; and
securing the strip first side to the flexible linking band and the strip second side to the endotracheal tube.

23. The method of claim 21, further including permanently affixing the bracket to the support strap band using at least one of stitches, sutures, and an adhesive.

24. The method of claim 21, wherein the central bridge portion comprises at least two laterally extending tabs, and wherein the flexible linking band is secured to the endotracheal tube by wrapping one of the at least two laterally extending extends around the endotracheal tube in a first direction relative to the central bridge and wrapping another of the at least two laterally extending tabs around the endotracheal tube in a direction substantially opposite the first direction relative to the central bridge.

25. The method of claim 21, wherein the bridge portion has a width that is greater than a width of the at least one laterally extending tabs and is connected to the upper bar portion at substantially rounded connections on opposing sides of the bridge portion.
